(19) United States
(12) Patent Application Publication
(10) Pub. No.: US 2006/0025741 A1
(43) Pub. Date: Feb. 2, 2006

(54) Highly deformable tampon

(75) Inventors: Thomas Ward Osborn III, Clifton, OH (US); Brian Kenneth Burgdorf, Norwood, OH (US); John Matthew Anast, Fairfield, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/900,950

(22) Filed: Jul. 28, 2004

(51) Int. Cl. A61F 13/20

(52) U.S. Cl. 604/385.18; 604/385.08

(57) ABSTRACT

A tampon is described that has an absorbent material positioned within a flexible overwrap. The absorbent materials can take many physical forms including particles, fibers, agglomerates, powders, gels, foams, beads and mixtures thereof. The tampon has properties that may be described by measurements of compression force, compression contact area, expulsion contact area, density, absorbency and retained absorbency.

### FIELD OF THE INVENTION

This invention relates to highly deformable tampons.

### BACKGROUND OF THE INVENTION

Generally catamenial tampons are rigid, have very low resiliency, and are small in their cross sectional dimension in order to provide insertion, wearing, and removal comfort. Generally, self-sustaining tampons are limited with respect to containment, capacity, and absorption rates. Self-sustaining tampons are generally self-sustaining into a cylindrical form and do not expand until contacted by fluid. Self-sustaining tampons range in size from about 0.8 cm to 2.0 cm in diameter and from 2 cm to 7 cm in length. The relatively small dimensions of self-sustaining tampons tend not to fill the vaginal cavity entirely, allowing menses to flow around or bypass them. As well, self-sustaining tampons have limited absorption rates and capacity due to their small surface areas and high density. In addition, self-sustaining tampons are considered by many women to be uncomfortable during use.

The tampon of the present invention has a highly deformable configuration. The present invention provides a solution to the drawbacks of the self-sustaining tampons in that its configuration fills the cross-section of the vagina, provides more containment, and has a greater capacity whereby establishing and maintaining a large void volume within the vagina. Comparatively, the tampon has a larger available surface area that provides good absorbency while being comfortable to wear, insert and remove.

### BACKGROUND ART

Two examples of one overwrap or bag are U.S. Pat. No. 3,812,856 issued to Robert Campbell Duncan and Darrel Dayfield Kokx relates to a hydro-dissociative agglomerate tampon and U.S. Pat. No. 3,815,601 relates to a catamenial aggregate absorbent body.

### SUMMARY OF THE INVENTION

This invention relates to a highly deformable tampon and an absorbent material positioned within a flexible overwrap. The material can take many physical forms including particles, fibers, agglomerates, powders, gels, foams, beads and mixtures thereof. The tampon has properties that may be described by measurements of compression force, compression contact area, expulsion contact area, density, absorbency and retained absorbency.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying drawing, in which:

FIG. 1 is a cut away view of the present invention to illustrate the tampon interior.

FIG. 2 is a diagram of pneumatic pressure device utilized in the Retained Absorbency Test.

### DETAILED DESCRIPTION OF THE INVENTION

As used herein the term “absorbency” refers to the amount or quantity of moisture or fluid retained by a material and is measured according to the absorbency test enclosed in the test methods below.

As used herein, the term “beads” refers to a piece or portion of material that can be a variety of geometrical configurations including but not limited to spherical, cylindrical, ovate, or rectangular, trapezoidal, and triangular with rounded edges. In general, the diameters of beads are from about 0.1 mm to about 6.0 mm. Typically, the diameter of beads may range in the size from about 1.0 mm to about 4.0 mm.

As used herein, “compression” refers to the process of pressing, squeezing, compacting or otherwise manipulating the size, shape, and/or volume of a material to obtain a tampon having a vaginally insertable shape. The term “compressed” refers to the state of a material or materials subsequent to compression. Conversely, the term “uncompressed” refers to the state of a material or materials prior to compression. The term “compressible” is the ability of a material to undergo compression.

“Density,” as used herein, is the tampon weight prior to preparation, or dry weight, divided by the volume and is measured according to the density test enclosed in the test methods below.

The term “digital tampon,” as used herein, refers to a tampon which is intended to be inserted into the vaginal canal with the user's finger and without the aid of an applicator. Thus, digital tampons are typically visible to the consumer prior to use rather than being housed in an applicator.

As used herein, the term “encased” refers to the positioning of an outer element in relation to an inner element whereby the outer element envelops, surrounds, enrobes, or otherwise covers the material as if in a case.

As used herein “hydrophilic” and “hydrophobic” have meanings as well established in the art with respect to the contact angle of a drop of water on the surface of a material. Thus, a material having a contact angle of greater than about 75 degrees is considered hydrophobic, and a material having a contact angle of less than about 75 degrees is considered hydrophilic. Absolute values of hydrophobocity/hydrophilicity are not generally important, but relative values are. Thus, the absorbent member of the tampon and the wicking overwrap of the present invention is more hydrophilic than the masking overwrap, and the masking overwrap is more hydrophobic than the absorbent member and the wicking overwrap.

The term “joined” or “attached” as used herein, encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

“Retained absorbency is amount of moisture retained by a material measured according to the retained absorbency test enclosed in the test methods below.

As used herein, a tampon has a “self-sustaining shape” when a tampon pledget has been compressed and/or shaped such that it assumes a general shape and size, which is vaginally insertable, absent external forces. It will be understood by one of skill in the art that this self-sustaining shape need not, and preferably does not persist during actual use of the tampon. That is, once the tampon is inserted and begins to acquire fluid, the tampon may begin to expand and may lose its self-sustaining form.

As used herein the term “tampon,” refers to any type of absorbent structure that is inserted into the vaginal canal or other body cavities for the absorption of fluid therefrom, to aid in wound healing, or for the delivery of active materials, such as medicaments, or moisture.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

The following terms are understood in relationship to the following graph generated in the Compression Time Test, which is further described in the test methods below. [image]
“Time Zero” is defined as the time at which 2 g-force is first reached upon initial compression and is represented as x₀ in the graph.
“Peak Compression Time” is defined as the time at which 600 g-force is first reached and is represented as y₀ in the graph.
“End of Protocol” is defined as the final data point in the protocol and is represented as z in the graph. z−y₀=40 seconds, as described in the method.
“Compression time” is defined as any time between Time Zero x₀ and Peak Compression Time y₀ and is represented as x₁ in the graph. x₀<x₁≦y₀
“Compression force” is defined as the force corresponding to a Compression Time y₁, the amount of force exerted on a tampon when used specifically in the context of the with Compression Contact Area Test or the Expulsion Contact Area Test.
“Relaxation time” is defined as any time between Peak Compression Time y₀ and End of Protocol z and is represented as y₁ in the graph. y₀<y₁≦z
“Relaxation force” is defined as the force corresponding to a Relaxation Time y₁.

### I. Tampon of the Present Invention

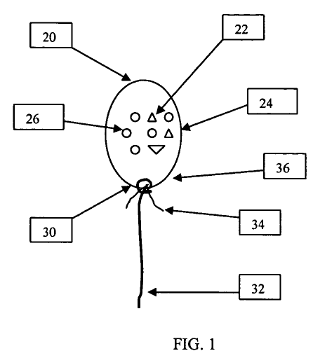

FIG. 1 illustrates a tampon 20 of the present invention. The present invention, however, is not limited to a structure having the particular configuration shown in the drawing.

At the center of the cut away view shown is the absorbent material 22, surrounding the material 22 is the overwrap 24 and at the periphery of the cut away is the overwrap 24. FIG. 1 depicts particles of absorbent foam-like material. The overwrap 24 is secured around the absorbent material 22 by a closure mechanism 30 that is proximate to the withdrawal end 36 of the tampon 20. FIG. 1 depicts the skirt portion 34, which extends beyond the closure mechanism 30 at the withdrawal end 36 and a withdrawal member 32 is attached to the overwrap 24.

The tampon 20 of the present invention may comprise an absorbent material 22 being positioned within a flexible overwrap 24; the tampon comprising a compression force of at least about 600 g in about 20 seconds. The tampon 20 may comprise a compression force of about 100 grams in at least about 20 seconds. Alternatively, the tampon 20 of the present invention may comprise an absorbent material 22 being positioned within a flexible overwrap 24, the tampon 20 comprising a compression force of at least about 600 g in about 20 seconds or longer and comprises a relaxation force of about 400 g or less in less than about 40 seconds after reaching a peak compression force of at least about 600 grams. The tampon 20 may reach a relaxation force of about 500 grams or less in less than 10 seconds after reaching a peak compression force of at least about 600 grams. Alternatively, the tampon 20 may reach a relaxation force of about 400 g or less in less than about 40 seconds after reaching a peak compression force of at least about 600 g.

The tampon 20 may have a compression contact area that increases by greater than about 1.0 in² (645 mm²) at a compression force of from about 100 grams to about 200 grams. The tampon 20 may have a compression contact area at least about 1.25 in² (806 mm²) at a compression force of about 600 grams or greater.

The tampon 20 may have an expulsion contact area that is at least about 1.2 in² (774 mm²) at a compression force of at least about 600 grams.

The tampon 20 may have a density of less than 0.2 g/cc and ratio of retained absorbency to absorbency at 1 psi at least about 0.5.
a. Overwrap:

The overwrap 24 in its pre-assembled state may be generally rectangular, but other shapes such as trapezoidal, triangular, hemispherical, chevron, hourglass shaped, and circular may also be acceptable. If the overwrap 24 is rectangular measures from about 1 inch (2.54 cm) to about 5 inches (12.7 cm) in length and from about 1 inch (2.54 cm) to about 5 inches (12.7 cm) in width. The overwrap 24 may be flexible and fluid permeable.

The overwrap 24 can be comprised of many materials including woven, non-woven materials, folded tissues, films (such as apertured formed thermoplastic films, apertured plastic films, reticulated thermoplastic films, and hydroformed thermoplastic films) or foams (such as porous foams and reticulated foams), that may comprise a blend of natural fibers, synthetic fibers or natural and synthetic fibers. The natural fibers may include but are not limited to rayon, cotton, wood pulp, flax, and hemp. Such acceptable types of rayon include GALAXY Rayon (a tri-lobed rayon structure) available as 6140 Rayon from Acordis Fibers Ltd., of Hollywall, England, SARILLE L rayon (a round cross-section fiber rayon), also available from Acordis Fibers Ltd. and SX 275-123 produced by Green Bay Nonwovens, Green Bay, Wis. The synthetic fibers can include but are not limited to fibers such as polyester (such as BIONELLE, a biodegradeable polyester), polyolefin, nylon, polypropylene, polyethylene, polyacrylic, vinyl polyacetate, polyacrylate, cellulose acetate or bicomponent fibers.

The overwraps 24 may have fibers with hydrophobic finishes, hydrophilic finishes, or combinations of hydrophobic or hydrophilic finishes. The fibers may be inherently hydrophilic or hydrophobic, or may be treated to provide such properties.

The overwrap 24 may include a material known as COROLIND nonwoven material, which is obtainable from BBA Nonwovens under the tradename PE HPC-2, code T23 FOR. The tampon 20 may have a nonwoven overwrap comprised of bicomponent fibers that have a polypropylene core surrounded by polyethylene manufactured by Vliesstoffwerke Christian Heinrich Sandler GmbH & Co. KG (Schwarzenbach/Saale, Germany) under the tradename SAS B31812000. The tampon 20 may comprise a nonwoven overwrap of a hydroentangled blend of 50% rayon, 50% polyester available as BBA 140027 produced by BBA Corporation of South Carolina, U.S.

The overwrap 24 can be comprised of a single layer of material, or may be layers of material. The overwrap 24 may be two or more layers of the same materials. Alternatively, the overwrap 24 may be two or more different materials. Multiple layers of overwrap 24 can be used. The outer layer of overwrap 24 may be less hydrophillic than the inner layer of overwrap 24. As well, the outer layer of overwrap 24 may have a more open structure or have larger capillaries than the inner layer of overwrap 24. The layers of overwrap 24 may be entangled, needle punched, hydroentangled, thermally bonded, or mixtures thereof. The layers of overwrap 24 may have similar chemical compositions or may be combinations that aid in the bonding of the two layers, such as, a rayon/polypropylene blend with polypropylene outer layer. The outerwrap 24 or portions of the outerwrap 24 may be mechanically altered to achieve a low modulus of stretch by processes such as, MICREXing, ring rolling process (as described in U.S. Pat. No. 4,107,364 issued to Sisson on Aug. 15, 1978, U.S. Pat. No. 4,834,741 issued to Sabee on May 30, 1989, U.S. Pat. No. 5,143,679 issued to Gerald M. Weber, et al. on Sep. 1, 1992, U.S. Pat. No. 5,156,793 issued to Kenneth B. Buell, et al. on Oct. 20, 1992, and U.S. Pat. No. 5,167,897 issued to Gerald M. Weber, et al. on Dec. 1, 1992) and the “SELFing” process (as described in U.S. Pat. No. 5,518,801 issued to Chappell, et al. on May 21, 1996.)

b. Absorbent Material:

The absorbent material 22 positioned within the flexible overwrap 24 may can take many physical forms including particles, fibers, agglomerates, powders, gels, foams or beads and mixtures thereof. Sizes of particles range from fine powders to about 8 mm. The dimensions of absorbent materials 22 are measured without a confining pressure. The absorbent materials 22 may be of any shape known in the art including but not limited to rods, cones, spheres, squares, chevrons, cylindrical, ovate, rectangular, trapezoidal, triangular or amorphous. The absorbent materials 22 may be comprised of one absorbent material or may include blends of absorbent materials. Blends may include different absorbent materials 22, different sized particles, or different shaped particles. One may include a blend of the same type of absorbent material 22 with different sizes and different shapes. Alternatively, one may include a blend of different type of absorbent materials 22 of the same size and same shape. The surface charges of the absorbent material 22 may be the same or different. The difference in surface charges may be altered via the addition of charged polymers to the outer surface of the particles or by using cationic absorbents. A quatinized chitosan may be used as the second material 26 and a HIPE foam may be used as the first material. Another example may include an alginate in combination with cotton and polyurethane foam. The fluid may be retained in the absorbent material 22 or may be retained in the interstitial spaces between the particles.

Suitable absorbent materials 22 include but are not limited to cotton (long fiber, short fiber, linters, T-fiber, card strips, and combe); rayon (such as GALAXY Rayon SARILLE L rayon both available from Acordis Fibers Ltd., of Hollywall, England); polysaccharides; comminuted wood pulp, which is generally referred to as airfelt; creped cellulose wadding; hydrogel polymer gelling agents; meltblown polymers including coform; chemically stiffened, modified or cross-linked cellulosic fibers; synthetic fibers including crimped polyester fibers, staple fibers; peat moss; absorbent foams (such as those disclosed in U.S. Pat. No. 3,994,298 issued to DesMarais on Nov. 30, 1976, U.S. Pat. No. 5,795,921 issued to Dyer, et. al both incorporated by reference herein,); capillary channel fibers (such as those disclosed in U.S. Pat. No. 5,356,405 issued to Thompson, et. al incorporated by reference herein); high capacity fibers (such as those disclosed in U.S. Pat. No. 4,044,766 issued Kaczmarzk et al. Aug. 30, 1977 incorporated by reference herein); superabsorbent polymers or absorbent gelling materials; (such as those disclosed in U.S. Pat. No. 5,830,543 issued to Miyake, et al incorporated by reference herein) absorbent foams; absorbent sponges; tissue including tissue wraps and tissue laminates; alginates; excipients (such as sodium starch glycolate sold under the name EXPLOTAB by Penwest Pharmaceuticals, Co., Patterson, N.J.), polymers or co-polymers of maleic anhydride (such as FIBERDRY by Camelot Technologies, Ltd. High River, AB, Canada), chitosans; cationic cellulosic polymers; polysaccarides or any equivalent material or combinations of materials, or mixtures of these.

Gel compositions may be used for the absorbent materials 22 such as those disclosed in U.S. Pat. No. 5,830,543 issued to Miyake, et al. Such gel compositions may include polyacrylamide super-absorbent premixed in water or glycerin to gel. The gelling agent may be water, glycerine, polyethylene glycols, or other materials that will gel the primary absorbent. Various compounds can be added to the gelling agents including foams, fibers or non-absorbent materials including but not limited to surfactants, salts of Na, Mg, Ca, etc or antibacterial agents or bacterial static agents, pH control agents or antioxidants including ascorbic acid.

The gel may absorb fluid and maintain an internal structure, so as to prevent the gel from squeezing out of the retaining layers during wear at body pressures and as it absorbs more menses. An example of this type of material is a fibrous absorbent gelling material with a non-gelling core, so that it maintains its fibrous structure while external surface gels. In this context, the elongated structure of the fibrous gel makes it particularly difficult to penetrate through small pores, voids or apertures of the retaining fabric, non-woven or film. It is believed that the longer the gel fiber is the lower the probability that the gel fiber will penetrate the overwrap 24. Some gel fibers include Oasis Fibers made by Technical Absorbents, U.K.

The gels may be combined with fibers. The length of the fibers or the size of the absorbent fiber may be varied. Longer fibers may be used. The fibers may range from 6 mm to 52 mm in length. A combination of longer and shorter fibers can be used, depending on gel strength and the probability that the gel fibers will penetrate through the overwrap 24 when gelled. Gel compositions may be typically combined with hydrophilic fibers such as, rayon, capillary fibers, fibers, polyethylene, polypropylene, polyester and mixtures thereof. It is believed that the fibers can help wick fluid into the gel core, as well as, keep the core open to more rapidly absorb fluid. Generally hydrophilic fibers may be used.

Various absorbent foams can be used as first and second absorbent materials. These foams may be relatively thin, collapsed, polymeric foam materials that, upon contact with aqueous body fluids, expand and absorb body fluid. For example the first and second material may comprise an open celled foam of the “High Internal Phase Emulsion” (HIPE) type or may also include “Thin after Drying” (TAD) HIPE absorbent foam. Such foam materials have cells and holes small enough to provide a high capillary absorptive pressure but large enough to prevent or minimize blockage by the insoluble components of blood and blood based liquids such as menses. Such suitable foams are disclosed in U.S. Pat. No. 5,387,207.

Types of absorbent foams that can be used are based on a wide range of polymers are available, including cellulose, cellulose acetate, cellulosic (rayon), styrene, polyolefins, polyvinyl halides, polyesters, polyvinylidene halides, polyurethanes, melamine/formaldehyde, polystyrene, polyacrylate, polyvinyl alcohol/formaldehyde, and many others. The common household sponge is often formed from viscose (dissolved cellulose) containing sodium sulfate, which is subsequently dissolved. Foams made from 2-hydroxyethyl methacrylate form hydrophilic sponges also. Polyolefin foams are probably the cheapest available. These are usually closed cell. Foams made from polyvinyl chloride (etc.) are narrowly used in specific applications that justify the cost. Chemically, these foams are comprised of an alkyl acrylate (e.g., 2-ethylhexylacrylate), divinyl benzene (DVB), ethyl styrene (eSTY), and optionally a second crosslinker derived from a diol or triol (e.g., hexanedioldiacrylate).

Suitable foams or combinations of foams may include those materials where the ratio of absorbencies measured at 1 psi and 0.25 psi (e.g. 1.0 psi absorbency/0.25 psi absorbency) are at least about 0.5.

HIPE absorbent foams can be prepared of an aqueous phase and an oil phase. The aqueous phase is prepared consisting of the ratios of materials as described in Table 1. The oil phase is prepared according to the monomer ratios described in Table 1, all of which include an emulsifier for forming the HIPE. The ingredients for the oil phase are purchased through Aldrich Chemical Co., Inc. (Milwaukee, Wis., USA), unless otherwise specified. The emulsifiers are also prepared according to the proportions described in Table 1 as a % by weight of total monomer mass. One particular emulsifier, diglycerol monooleate (DGMO; Grindsted Products; Brabrand, Denmark) comprises approximately 81% diglycerol monooleate, 1% other diglycerol monoesters, 3% polyglycerols, and 15% other polyglycerol esters, imparts a minimum oil phase/aqueous phase interfacial tension value of approximately 2.5 dyne/cm and has a critical aggregation concentration of approximately 2.9 wt %. The monomers plus the emulsifiers make up the oil phase.

To form the HIPE, the oil phase is weighed into a high-density polyethylene cup with vertical sides and a flat bottom. The internal diameter of the cup is 3″ and the height of the cup is 4.75″ (these dimensions being primarily for convenience). The aqueous phase is placed in a Lab Glass (Vineland, N.J., USA) jacketed addition funnel Model LG-8432-100 and held at a pour temperature of about 65° C. The contents of the plastic cup are stirred using a Caframo RZR50 (Caframo Limited, Wiarton, Ontario, Canada) stirrer with a six-bladed stirrer rotating at about 300 rpm (adjustable by operator as needed). At an addition rate sufficient to add the aqueous phase in a period of about 2 to 5 minutes, the aqueous phase is added to the plastic cup with constant stirring. The cup is moved up and down as needed to stir the HIPE as it forms so as to incorporate all the aqueous phase into the emulsion.

Next, the HIPE foam is polymerized and cured. The HIPE in the 3″ plastic cups are capped and placed in an oven set at the cure temperature outlined in Table 1 and a cure time of 18 hours to provide polymeric HIPE foam. Some formulations may require substantially less time for curing (e.g. continuous process), but 18 hours provides enough time for all formulations to cure.

The HIPE may be washed and dewatered after the polymerization and curing step. The cured HIPE foam is removed from the cup as a cylinder 3″ in diameter and about 4″ in length. The foam at this point has residual aqueous phase (containing dissolved emulsifiers, electrolyte, initiator residues, and initiator) about 50-60 times (50-60) the weight of polymerized monomers. The foam is sliced on a Hobart Model 1612 meat slicer (Hobart Corp, Troy, Ohio, USA) to give circular pieces about 0.5 mm to about 15 mm in thickness. These pieces are washed in distilled water and compressed to remove the water 2 to 4 times. In some cases they may be washed and compressed further in 2-propanol about 3 to 4 times. The pieces are then dried in an oven at the cure temperature specified in Table 1 for 18 hours. In some cases, the foams collapse upon drying and must be freeze-dried from the water-swollen state to recover fully expanded foams. Various shapes and sizes of foams may be prepared similarly by use of appropriately shaped vessels in which the HIPE is cured and/or appropriate cutting or shaping. The process for preparing the foams of the present invention may also be a one such as that described in U.S. Pat. No. 5,149,720, issued Sep. 22, 1992 to DesMarais et al. or copending U.S. patent application Ser. No. 08/370,694, filed by DesMarais on Jan. 10, 1995, the disclosure of each of which is incorporated by reference.

The foam pieces are then run through an Imperia SP150 (Turin, Italy) pasta maker to chop them into smaller pieces of varying widths and sizes, which are then comprised into the final tampon 20.

TABLE 1

| | Oil Phase | | | | | | | | | | | Aqueous | | | |
|---|---|---|---|---|---|---|---|---|---|---|---|---|---|---|---|
| | Monomers | | | | | | | | Emulsifiers | | | Phase | | Aqueous: | Cure |
| Condi- | % | % | % | % | % | % | % | % | % | % | % | % | % | Oil | Temp |
| tion | DVB₅₅ | EHA | HDDA | STY | IOA | EHMA | EGDMA | NPDMA | DGMO | PGS | DTDMAMS | CaCl₂ | KPS | ratio | (° C.) |
| A | 15.1 | 59.9 | | 25 | | | | | 6 | | | 10 | 0.05 | 18:1 | 85 |
| B | 25.5 | 57.5 | 12 | 5 | | | | | 6 | | | 4 | 0.05 | 30:1 | 75 |
| C | | 35 | 35 | | 30 | | | | 5 | | | 4 | 0.05 | 25:1 | 65 |
| D | | 40 | | | | 42 | 18 | | | 6 | 1 | 4 | 0.05 | 30:1 | 65 |
| E | | 42 | | | | 40 | 18 | | | 6 | 1 | 4 | 0.05 | 25:1 | 65 |
| F | | 70 | | 10 | | | 20 | | | 6 | 1 | 4 | 0.05 | 25:1 | 65 |
| G | | 45 | | | | 36 | | 19 | | 6 | 1 | 4 | 0.05 | 25:1 | 65 |
| H | | 40 | | | | 41 | 12 | 7 | | 6 | 1 | 4 | 0.05 | 25:1 | 65 |
| * DVB₅₅ = divinyl benzene of 55% purity obtained from Dow Chemical of Midland, MI plus styrene; EHA = 2-ethylhexyl acrylate; HDDA = 1,6-hexanediol diacrylate; STY = styrene; IOA=; EHMA = 2-ethylhexyl methacrylate; EGDMA = ethylene glycol dimethacrylate; | | | | | | | | | | | | | | | |
| # NPDMA=; DGMO = diglycerol monooleate; PGS = polyglycerol succinate, which is formed from an alkyl succinate and glycerol and triglycerol; DTDMAMS = ditallow dimethyl ammonium methyl sulfate; CaCl₂ = calcium chloride; KPS = potassium persulfate; | | | | | | | | | | | | | | | |

c. Optional Components

i. Non-Absorbent Material

Some non-absorbent materials may be blended with the absorbent material 22 including but are not limited to silica or plastic beads. Other non-absorbent material may include polyethylene, polypropylene, polyester, and polyesters. Such non-absorbent material can change and impart properties to the tampon 20 structure such as facilitating spread of the tampon 20 within the vaginal cavity and delivery of medicines.

ii. Lubricant:

In addition, lubricant may be used on the outer overwrap 24 to help the tampon 20 spread over the vaginal surface. Lubricants such as “KY Jelly,” glycerin or personal lubricants commonly used in the vagina may be used. The amount of the lubricant can be varied to provide optimal spreading upon insertion. Non-aqueous lubricants, such as silicones and dimethicones can be added.

iii. Closure Mechanism:

The closure mechanism 30 can be any of the known variety including sewing, gluing, tying with a string, heat sealing or ultrasonic bonding. This could include gathering, such as bringing together of the overwrap 24 at a longitudinal end to form a closure of the overwrap 24 at that end, a closure mechanism 30 which is omni-directionally gathered radially inwardly, as if drawn by a drawstring.

iv. Skirt Portion:

Optionally, the tampon 20 of the present invention may include a skirt portion. A skirt portion may be formed when the overwrap 24 is closed such that at least a portion of the overwrap 24 extends below the closure mechanism 30 of the structure. Typically, the overwrap 24 can extend from about 2 mm to about 30 mm beyond the closure mechanism 30 proximate to the withdrawal end of the withdrawal end of the tampon 20. Both the compressed absorbent member and skirt portion of the overwrap 24 may reside either entirely, substantially or partially within the vaginal cavity of the wearer during use of the tampon 20. Only the withdrawal member 32 may reside externally to the orifice of the vagina.

v. Withdrawal Member

Optionally the tampon 20 of the present invention will comprise one or more a withdrawal members 32 that are joined to the tampon 20 for removal of the tampon after use. The withdrawal member 32 may be joined to the overwrap 24 at the withdrawal end 26 of the tampon 20 by any suitable manner known in the art including sewing, adhesive attachment, knotting, or punching the withdrawal member 32 through the bag and looping it around the tampon 20 to form a knot or a combination of known bonding methods including the method disclosed in currently pending, commonly assigned U.S. patent application Ser. No. 10/610,075 filed Jun. 30, 2003 entitled “Method and Apparatus for Cord Attachment” to Sargent, et al. Any of the withdrawal members 32 currently known in the art may be used including a ribbon, loop, tab, or the like.

vi. Applicator

The tampon 20 of the present invention may be inserted digitally or through the use of an applicator. Any suitable applicator may also be used for insertion of the tampon 20 of the present invention including the “tube and plunger” type and “compact” type applicators. The applicators may be plastic, paper, or other suitable material.

II. Process of Making

While several methods of making the tampon 20 of the present invention would be apparent to one of skill in the art in light of the disclosure herein, following is a description of one method of making a tampon 20 of the present invention.

The process for making a tampon 20 comprises the steps of providing an absorbent material 22. A flexible overwrap 24 is provided. The absorbent material 22 is positioned within a flexible overwrap 24. Optionally, a closure mechanism 30 and withdrawal member 32 is provided.

III. Test Methods

### Absorbency Test

The Absorbency test that is performed on the tampon samples is obtained at 0.25 psi by Syngyna Method found in FDA 21 CFR Ch. 1. The Standard Syngyna Test is as follows:

An unlubricated condom, with tensile strength between 17 Mega Pascals and 30 Mega Pascals is attached to the large end of a glass chamber with a rubber band and pushed through the small end of the chamber using a smooth, finished rod. The condom is pulled through until all slack is removed. The tip of the condom is cut off and the remaining end of the condom is stretched over the end of the tube and secured with a rubber band. A preweighed (to the nearest 0.01 gram) tampon is placed within the condom membrane so that the center of gravity of the tampon is at the center of the chamber. An infusion needle (14 guage) is inserted through the septum created by the condom tip until it contacts the end of the tampon. The outer chamber is filled with water pumped from a temperature-controlled waterbath to maintain the average temperature at 27±1 C. The water returns to the waterbath. Syngyna fluid (10 grams sodium chloride, 0.5 gram Certified Reagent Acid Fushsin, 1,000 milliliters distilled water) is then pumped through the infusion needle at a rate of 50 milliliters per hour. The test shall be terminated when the tampon is saturated and the first drop of fluid exits the apparatus. (The test result shall be discarded if fluid is detected in the folds of the condom before the tampon is saturated). The water is then drained and the tampon is removed and immediately weighed to the nearest 0.01 gram. The absorbency of the tampon is determined by subtracting its dry weight from this value. The condom shall be replaced after 10 tests or at the end of the day during which the condom is used in testing, whichever occurs first.

### Retained Absorbency Test

Testing Equipment:

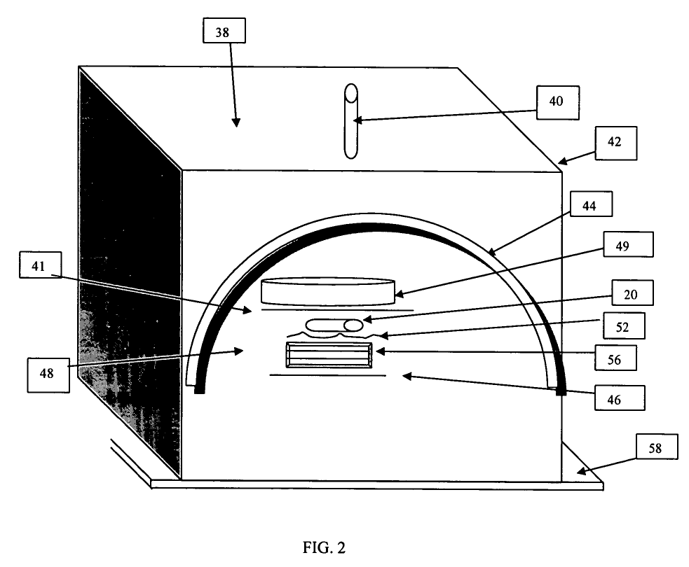

The retained absorbency test utilizes a calibrated balance (Mettler PG802) manufactured by Mettler Instrument Corp., NJ that is accurate to 0.01 g. The retained absorbency test utilizes a pneumatic pressure device, which is shown in FIG. 2. The pneumatic pressure device is comprises a rigid housing 42 with a conformable film 44, a piston 40 and a Magnehelic pressure device (not shown). The piston 40 is connected the rigid housing 42 and a Magnehelic pressure gauge. Custom Tooling Company, Ohio, manufactures the conformable film 44. The Magnehelic pressure gauge is accurate to 0.06 psi manufactured by Dwyer Instruments, Inc, Michigan.

Test Protocol:

First, a layered mass is formed from a layer of film 46 is covered by 15 filter papers 56 that is covered by a sheet of nonwoven 52. The film is SEALWRAP manufactured by Borden packing, MA. The filter papers 56 used #632 that are 5″×5″ manufactured by Ahlstrom, Ohio. The nonwoven 52 is 27 g/m² comprising carded polypropylene manufactured by BBA, Old Hickory, Tenn. under the product code FPN332.

The tampon 20 is placed on top of the film 48, filter paper 56, and nonwoven 52 and a layer of SEALWRAP film 41 is placed on top of the tampon 20 and a layer of polyurethane foam 46 is placed on top of the SEALWRAP 41. The polyurethane foam 49 utilized has a compression modulus of 0.3N/cm2, and a caliper of 12.5 mm at 0 psi, 2.5 mm at 1.0 psi and 2.1 mm at 1.5 psi.

Pressure is applied to the tampon 20 and released and then the weight of the tampon 20 is taken. Pressure is applied to the tampon 20 at 1.0 psi and held for 6 seconds, and then the pressure is released. The weight of the tampon 20 is taken on the calibrated balance. Next, pressure is applied to the tampon 20 is applied at 1.5 psi and hold for about 6 seconds. The weight of the tampon 20 is taken on a scale. The difference between this weight and the dry weight is designated as retained absorbency at the applied pressure.

### Compression Time Test

The compression time test measures force and time as a tampon is compressed to reach a peak compression force of 600 g and records the force and time for relaxation to occur.

Testing Hardware And Software:

The compression test utilizes a MTS Alliance RT/1 tensile tester equipped with Testworks 4.04D software, both manufactured by MTS Systems Corporations, Eden Prairie, Minn. The MTS Alliance RT/1 is configured with two platens, an upper platen and a lower platen. These platens are circular with a central radius. The lower platen is 10.0 cm in diameter and the upper platen is 5.0 cm in diameters. Prior to the testing, the upper platen and lower platen are separated by 10 cm to easily accommodate placement of tampons of various sizes on the lower platen. The upper and lower platens are leveled and arranged parallel to each other.

MICROSOFT EXCEL 2000 software manufactured by the Microsoft Corporation, Seattle, Wash. is used to normalize and graph the data generated.

Sample Preparation:

The tampon sampled is a standard size for human use, typically 20-70 mm in length and 8-20 mm wide. The tampon sample is removed from the carton and conditioned in the individual tampon wrapper and, if non-digital, within the applicator at 72° F. and 50% relative humidity for 168 hours prior to testing. To run the compression test, the tampon is removed from any wrapper and is ejected from the applicator quickly (less than 2 seconds) or, if digital, it is simply removed from the wrapper. The tampon sample is visually centered along the central axis of the lower platen. The tampon sample is so arranged on the lower platen such that withdrawal means should not affect the compression testing. Typically, this is accomplished by placing the longitudinal axis of the tampon sample parallel to the surface plane of the lower platen. The longitudinal axis of the tampon is the vertical when the tampon sample is hung vertically by a portion of the withdrawal means. The compression testing should be initiated within 5 to 10 minutes of ejecting the tampon from the applicator or removing the tampon from the wrapper, if no applicator is present.

Test Protocol:

All the channels on the machine are zeroed. The compression program is set-up such that the strain rate is set at 25 mm/min and data acquisition at 25 Hz. The upper platen is manually positioned 5 mm above the outer surface of the tampon. The compression program is set up such that the upper and lower platen compress the tampon sample at a rate of strain of 25.0 mm/min until the applied stress reaches 600 g-force, whereupon the platen positions are held for 40 seconds. The compression program is run and data is generated.

The data is exported to MICROSOFT EXCEL software. The data is plotted on a graph, where load (g-force) versus time (sec). To normalize the data, time zero is defined as the point where the 2 g-force is reached. The compression time is shown on the graph as the time that elapsed prior to the tampon sample reaching the 600-g compression force.

### Compression Contact Area Test and Expulsion Contact Area Test

The Compression Contact Area Test and the Expulsion Contact Area test use a scanner and imaging software to measure the area of the tampon that contacts the scanner at full expulsion and 50% of expulsion while under a compression force.

Testing Hardware and Software:

The Compression Contact Area Test utilizes a MTS Alliance RT/1 tensile tester equipped with Testworks 4.04D software, both manufactured by MTS Systems Corporations, Eden Prairie, Minn. The MTS Alliance RT/1 is configured with two platens, an upper platen and a lower platen. These platens are circular with a central radius. The lower platen is 10.0 cm in diameter the upper platen is 5.0 cm in diameter. An Epson 1640 SU scanner, with lid removed, is placed onto the lower platen so that it is centered below the upper platen surface. Prior to the testing, the upper platen and the surface of the scanner are separated by 10 cm to easily accommodate placement of various sizes of tampons on the scanner surface. The upper platen and the surface of the scanner should be leveled to ensure that they are parallel prior to each test series. The scanner settings are Exposure 0, Gamma 1.8, Highlights 245, Shadows 7, and Resolution 300 dpi.

ImageJ software, version 1.28 used to binarize the scanner images and calculate the contact area, was authored by Wayne Rasband at the Research Services Branch, National Institute of Mental Health, Bethesda, Md., USA.

### Compression Contact Area

Sample Preparation:

The tampon sample used is a standard size for human use, typically 20-70 mm in length and 8-20 mm wide. The tampon sample is removed from the carton and conditioned within the individual tampon wrapper and, if non-digital, within the applicator at 72° F. and 50% relative humidity for 168 hours prior to testing. The tampon is removed from any wrapper and is ejected from the applicator quickly (less than 2 seconds) or if, digital, it is simply removed from the wrapper. The tampon sample is visually centered on the surface of the scanner. The tampon sample is so arranged on the lower platen such that withdrawal means should not affect the compression testing. Typically, this is accomplished by placing the longitudinal axis of the tampon sample parallel to the surface plane of the scanner. The longitudinal axis of the tampon is the vertical axis when the tampon sample is hung vertically by portion of the withdrawal means. The compression testing should be initiated within 5 to 10 minutes of ejecting the tampon from the applicator or removing from the wrapper, if no applicator is present.

Test Protocol:

All the channels on the MTS machine are zeroed. The compression program is set-up such that the strain rate is set at 25-mm/min and data acquisition at 25 Hz. The upper platen is manually positioned 5 mm above the outer surface of the tampon. The compression program is set up such that the upper platen and surface of the scanner compresses the tampon sample at a rate of strain of 25.0 ml/min to a stress applied of 100 g-force, whereupon the platen positions are held for 1 minute and the tampon scanned and image captured. The compression program is then run to a stress of about 200 g-force, whereupon the platen positions are held for 1 minute and a scan is again taken and captured. The compression, hold and scan process is repeated for stresses of 300, 400, 600, 800, and 1000 g-force. The compression program is run. The tampon samples are scanned and images are captured at the hold times at 100 g, 200 g, 300 g, 400 g, 600 g, 800 g, and 1000 g forces by the Epson 1640SU scanner.

After, the compression program is complete, the scanned images of the tampon samples are exported to ImageJ software. For each image, the user defines a brightness threshold to select the outer perimeter of the tampon contacting the surface of the scanner. For example, during testing one of three threshold values were used depending on the brightness of the product's overwrap. For the brightest products a threshold of 235 was used, for all others a value of 215 was used with the exception of a single product, which had a semi-transparent overwrap for which a value of 175 was used. Once the user defines the brightness threshold, the image is binarized based on the brightness threshold defined by the user. The area of the images with brightness values greater than the defined brightness threshold is measured and is reported in mm². This measured area is the compression contact area.

### Expulsion Contact Area

Sample Preparation:

The tampon sample used is a standard size for human use, typically 20-70 mm in length and 8-20 mm wide. The tampon sample is removed from the carton and conditioned with the individual tampon wrapper at 72° F. and 50% relative humidity for 168 hours prior to testing. Remove the sample from the wrapper, expel and lock the tampon samples to “50% expulsion.” “50% expulsion” refers to halfway point in the insertion process, for applicators with closed ends, it is the halfway point between the point where the tampon is completely enclosed by the applicator and the point where the tampon is fully expelled from the applicator. The tampon sample can be locked at 50% expulsion by piercing a tack, or similar means through the outer and inner tube of the applicator. Place tampon and applicator into a rigid fixture on the MTS Alliance RT/1 where the tampon end of applicator is directed downward, perpendicular to the imaging surface of scanner.

Test Protocol:

All the channels on the MTS machine are zeroed. The compression program is set-up such that the strain rate is set at 25-nm/min and data acquisition at 25 Hz. The upper platen is manually positioned so that the outer surface of the tampon facing the scanner surface is 5 mm from the scanner surface. The compression program is set up such that the upper platen and the upper surface of the scanner compress the tampon sample at a rate of strain of 25.0 nm/min to a stress applied of 100 g-force, whereupon the platen positions are held for 1 minute, then the tampon is scanned and the image is captured; the compression program is then run to a stress of about 200 g-force, whereupon the platen positions are held for 1 minute, then the tampon is scanned and the image is captured. The compression, hold, scan and capture image process is repeated for stresses of 300, 400, 600, 800, and 1000 g-force. The compression program is run. The tampon samples are scanned and images are captured during the hold times at 100 g, 200 g, 300 g, 400 g, 600 g, 800 g, and 1000g forces by the Epson 1640 SU scanner.

After, the compression program is complete, the scanned images of the tampon samples are exported to ImageJ software. For each image, the user defines a brightness threshold to select the outer perimeter of the tampon contacting the surface of the scanner. For example, during testing one of three threshold values were used depending on the brightness of the product's overwrap. For the brightest products a threshold of 235 was used, for all others a value of 215 was used with the exception of a single product, which had a semi-transparent overwrap for which a value of 175 was used. Once the user defines the brightness threshold, the image is binarized based on the brightness threshold defined by the user. The area of image brightness values greater than the brightness threshold is measured and reported in mm². This measure area is the expulsion contact area.

### Tampon Volume and Density Test

Testing Hardware and Software:

The tampon volume test utilizes a PG 802 calibrated mass balance manufactured Mettler Toledo, Inc. Columbus, Ohio and a Bruker Biospin Advance MRI spectrometer operating at 200.4 Mhz (4.7 tesla) with the Bruker Paravision 2.1.1 software both manufactured by Bruker Biospin, Etlingen, Germany. The tampon sample images are scaled by MatLab software version 6.5.0 manufactured by The Mathworks, Inc. Nattick, Mass. and segmented by Slice-o-matic software version 4.2-R7c manufactured by TomoVision, Montreal Canada.

Sample Preparation:

The tampon sample used is a standard size for human use, typically 20-70 mm in length and 8-20 mm wide. If tampon samples are housed in applicators, the tampon samples should be ejected from the applicator. Each tampon was prepared by removing the string without cutting or disturbing the main tampon body or closure. The product was weighed using a calibrated mass balance. The tampon is then prepared for imaging by spraying the surface as uniformly as possible with a spray canola oil (Marsh brand Cooking Spray, Indianapolis, Ind.) at a level of 10-30 mg/cm² of surface area. For current tampons of approximate density of 0.30-0.50 g/cm³, the product can be laid on a flat surface and gently rolled while applying the spray. For the other tampon samples where contact with a surface would distort the shape, the tampon sample was suspended by a thread around the closure and prepared by spraying the product while gently rotating it. The scanning should be initiated within 20 minutes of ejecting the tampon from the applicator.

Test Protocol:

The tampon is placed in the bore of a Bruker Biospin Advance MRI spectrometer. A 3D spin echo pulse sequence (TR=400 ms, TE=6 ms) is used with a field of view of 8 cm×8 cm×8 cm and a matrix of 128×128×64. For current tampons of approximate density of 0.30-0.50 g/cm³, the products were laid on the flat gantry while imaging. For the other products where contact with a surface would distort the shape, the tampon was suspended by a thread around the closure, so the tampon does not contact any surface.

The tampon samples are scanned and images are collected of the tampon products outline. The images are downloaded to MatLab software, and the downloaded images are scaled from 32 bit to 16 bits using MatLab software.

The scaled images were downloaded into Slice-o-matic software where the images are segmented using the Threshold tool with a setting of 767. Portions of the tampon outline within the images which were not fully defined by the use of oil are segmented by manually interpolating from nearby portions of the image using the Slice-o-matic Edit tool. The interior of the tampon outlines are then segmented with the Flood Fill tool. The Shell tool is utilized to obtain the volume of the tampon samples from the segmented images.

The tampon sample's volume and surface area are calculated by the Shell tool and are recorded. The tampon weight in grams without the extraction cord attached and prior to adding the image contrast oil, is divided by the volume in cc as determined by the Volume Test to determine the density of the tampon.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon comprising:
an absorbent material being positioned within a flexible overwrap;
said tampon comprises a compression force of at least about 600 grams in at least about 20 seconds.

2. The tampon according to claim 1 wherein said tampon comprises a relaxation force of about 400 grams or less in less than 40 seconds after reaching a compression force of at least about 600 grams.

3. The tampon according to claim 1 wherein said tampon comprises a relaxation force of less than about 500 grams in less than 10 seconds after reaching a compression force of at least about 600 grams.

4. The tampon according to claim 1 wherein said tampon comprises a compression force of at least about 100 g in at least about than about 20 seconds.

5. The tampon according to claim 1 wherein said tampon comprises a compression contact area; said compression contact area increases by greater than about 645 mm² at a compression force of between about 100 grams to about 200 grams.

6. The tampon according to claim 1 wherein said tampon has an expulsion contact area greater than 774 mm² at a force at least about 600 grams.

7. The tampon according to claim 1, wherein said absorbent material is blended with a non-absorbent material selected from the group consisting of silica, plastic, polyethylene, polypropylene, polycarbonate, and polyesters or mixtures thereof.

8. The tampon according to claim 1 wherein, said absorbent material is selected from the group consisting of cotton; rayon; polysaccharides; comminuted wood pulp; creped cellulose wadding; hydro gel polymer gelling agents; meltblown polymers; carboxy-methyl cellulose; cross-linked carboxylmethyl cellulose; polyacrylimides; polyacrylates crimped polyester fibers; staple fibers; peat moss; absorbent foams; absorbent sponges; tissue wraps; laminates; alginates; excipients, chitosans; cationic cellulosic polymers; polysaccharides or mixtures thereof.

9. The tampon according to claim 1 wherein said tampon comprises a compression contact area; said compression contact area is at least about 806 mm² at a compression force of about 600 grams.

10. A tampon comprising:
an absorbent material being positioned within a flexible overwrap;
said tampon comprises a compression force of at least about 600 g in at least about 20 seconds;
and said tampon comprises a relaxation force of less than about 400 grams in less than about 40 seconds after reaching a compression force of at least about 600 grams.

11. The tampon according to claim 10 wherein said tampon comprises a compression force of at least about 100 grams in at least about 20 seconds.

12. The tampon according to claim 10 wherein said tampon comprises a compression contact area; said compression contact area increases by an area greater than about 645 mm² at a compression force of from about 100 grams to about 200 grams.

13. The tampon according to claim 10, wherein said absorbent material is blended with a non-absorbent material selected from the group consisting of silica, plastic, polyethylene, polypropylene, polycarbonate, and polyesters are mixtures thereof.

14. The tampon according to claim 10 wherein, said absorbent material is selected from the group consisting of cotton; rayon; polysaccharides; comminuted wood pulp; creped cellulose wadding; hydro gel polymer gelling agents; meltblown polymers; carboxy-methyl cellulose; cross-linked carboxylmethyl cellulose; polyacrylimides; polyacrylates crimped polyester fibers; staple fibers; peat moss; absorbent foams; absorbent sponges; tissue wraps; laminates; alginates; excipients, chitosans; cationic cellulosic polymers; polysaccharides and mixtures of thereof.

15. A tampon comprising:
an absorbent material being positioned within a flexible overwrap; said tampon comprising a density of less than 0.2 g/cc and ratio of retained absorbency to absorbency at 1 psi of at least about 0.5.

16. The tampon according to claim 15 wherein said tampon comprises a peak compression force of at least about 600 grams in about 20 seconds.

17. The tampon according to claim 15 wherein said tampon comprises a relaxation force of less than about 400 grams in less than 40 seconds after reaching a compression force of at least about 600 grams.

18. The tampon according to claim 15 wherein said tampon comprises a compression contact area; said compression contact area increases by greater than 645 mm² at a compression force of between about 100 grams and 200 grams.

19. The tampon according to claim 15, wherein said absorbent material is blended with a non-absorbent material selected from the group consisting of silica, plastic, polyethylene, polypropylene, polycarbonate, and polyesters and mixtures thereof.

20. The tampon according to claim 15 wherein, said absorbent material is selected from the group consisting of cotton; rayon; polysaccharides; comminuted wood pulp; creped cellulose wadding; hydro gel polymer gelling agents; meltblown polymers; carboxy-methyl cellulose; cross-linked carboxylmethyl cellulose; polyacrylimides; polyacrylates crimped polyester fibers; staple fibers; peat moss; absorbent foams; absorbent sponges; tissue wraps; laminates; alginates; excipients, chitosans; cationic cellulosic polymers; polysaccharides and mixtures thereof.
